(19) United States
(12) Patent Application Publication
(10) Pub. No.: US 2024/0009289 A1
(43) Pub. Date: Jan. 11, 2024

(54) THERAPEUTIC VACCINE COMPRISING A SPECIFIC ANTIGEN OF A DISEASE THAT DOES NOT AFFECT THE CENTRAL NERVOUS SYSTEM AND NANOPARTICLES, AND USE OF THE VACCINE

(71) Applicant: Vaxinano, Loos (FR)

(72) Inventors: Olivier Betbeder, Houplin-Ancoisne (FR); Phillippe Loiseau, Gif-Sur-Yvette (FR); Juliane Sousa Lanza, Marseille (FR); Sandrine Cojean, Bonneuil-Sur-Marne (FR)

(21) Appl. No.: 18/251,658

(22) PCT Filed: Sep. 24, 2021
(86) PCT No.: PCT/FR2021/051655

§ 371 Date: May 3, 2023

(30) Foreign Application Priority Data
Nov. 4, 2020 (FR) 2011304

(51) Int. Cl. A61K 39/008; A61P 31/04; A61K 9/00

(52) U.S. Cl. CPC A61K 39/008; A61P 31/04; A61K 9/0043; A61K 2039/552

(57) ABSTRACT

A therapeutic vaccine for treating individuals who are carriers of a disease or pathogen that does not affect the brain, such as leishmaniasis, cancer or any pathogenic infection, comprises at least one cationic nanoparticle comprising a cationic polysaccharide core and a specific antigen, wherein the antigen is specific to the pathogen or disease.

### CROSS-REFERENCE TO RELATED APPLICATIONS

This application is a national phase entry under 35 U.S.C. § 371 of International Patent Application PCT/FR2021/051655, filed Sep. 24, 2021, designating the United States of America and published as International Patent Publication WO 2022/096792 A1 on May 12, 2022, which claims the benefit under Article 8 of the Patent Cooperation Treaty to French Patent Application Serial No. 2011304, filed Nov. 4, 2020.

### TECHNICAL FIELD

The present disclosure relates to the field of immunotherapeutic compositions used as therapeutic vaccines. It relates more particularly to a therapeutic vaccine for treating diseases that do not affect the brain, such as leishmaniasis.

### BACKGROUND

Therapeutic vaccination consists of inducing an immune response, when such response is insufficient to allow spontaneous healing. The activation of the immune system requires on the one hand that it recognize the intruder and, on the other hand, that it consider the intruder to be dangerous, and then that an appropriate response is induced, both in terms of specificity and intensity.

The therapeutic strategy is very different from that of prophylactic vaccination. Prophylactic vaccination consists of presenting an antigen to the immune system so as to induce a rapid, strong response when the organism comes in contact with the same antigen again (memory response). Therapeutic vaccination is based on the fact that the pathogen is already present in the organism. This is because during an infection, the immune defenses are often overwhelmed by infectious proliferation or hindered by internal control mechanisms, which prevents an effective immune response against the pathogen. The therapeutic vaccine will allow amplification of the specific immune response.

The main therapeutic vaccines developed to date have the objective of treating cancer. They generally consist of injecting the tumor antigen and/or injecting differentiated dendritic cells and/or T lymphocytes.

Therapeutic vaccines are of interest since they constitute an effective therapeutic solution for individuals who already carry the disease. They also make it possible in the case of certain pathologies to generate immune responses not induced by conventional prophylactic vaccines.

Leishmaniasis is a parasitic disease that affects many species of mammals and, in particular, humans and dogs. The number of household dogs affected by canine leishmaniasis is estimated at 2.5 million, but more generally distributed in 70 countries worldwide, and located on all continents. The incidence of this disease is also strong in humans, with about 2 million new cases per year, found in more than 80 countries worldwide. This disease is the result of an infection by an intracellular parasite of the genus Leishmania. This parasite is transmitted by a vector diptera insect, phlebotomines. The geographical distribution of the disease therefore depends on the distribution of phlebotomines and its reservoirs: mammals. Leishmania infantum is an intra-macrophage parasite responsible for the disease in Europe, in the Mediterranean basin and in South America. Contamination travels through an insect bite on the individual. The disease can also be transmitted from the mother to the child.

Parasites of the Leishmania genus infect host cells and divert the intracellular traffic mechanism in order to maintain their parasitophorous vacuole wherein they proliferate.

The prior art reports developments of therapeutic formulae for the treatment and/or diagnosis of leishmaniasis. For example, ES2205059 discloses a pharmaceutical composition comprising an immunogenic part of a Leishmania antigen. However, none of the therapeutic vaccine tests against leishmaniasis has proved effective to date.

In addition, the compositions proposed in the prior art comprise all vaccinal adjuvants, which are necessary for their effectiveness, but whose side effects are widely documented.

Finally, the most widespread current treatment of leishmaniasis is carried out by chemotherapy. This treatment is expensive, nonspecific, toxic and can induce the appearance of chemoresistance. The molecules known for the treatment of leishmaniasis in dogs are meglumine antimoniate (GLUCANTIME®) and allopurinol, often used in combination.

In this context, there is a need to have an effective treatment that does not induce any resistance for infectious diseases due to a pathogen that does not infect the central nervous system such as leishmaniasis, but also improves the efficacy of therapeutic vaccines in general, in particular, in the treatment of cancer.

### BRIEF SUMMARY

A therapeutic vaccine, without adjuvants, intended for administration in an individual carrying a disease or a pathogen that does not reach the central nervous system has been developed. In particular, the therapeutic vaccine may be used as treatment against leishmaniasis or cancer.

Thus, the present disclosure relates to a therapeutic vaccine for treating a living being who carries a disease or pathogen that does not affect the central nervous system, comprising at least one cationic nanoparticle consisting of a cationic polysaccharide nucleus and at least one antigen specific to the disease or the pathogen that does not affect the central nervous system.

In a particular embodiment, the present disclosure relates to a therapeutic vaccine intended for the treatment of leishmaniasis wherein the antigen is specific to the species Leishmania.

Quite surprisingly, it has been demonstrated that administration to an organism, already carrying a disease or pathogen that induces harm to metabolic systems, such as leishmaniasis, from an immunotherapeutic composition comprising nanoparticles and an antigen specific to the pathogen or the disease makes it possible to treat the individual. During infection by a pathogen, this type of treatment is also likely to induce specific long-term immunity, making it possible to avoid subsequent reinfection by the same pathogen. Administering the vaccine while the individual is already carrying the disease makes it possible to effectively enlist the immune defenses of the individual and to guarantee healing by eliminating the pathogen and long-term immunity by putting memory T cells in place.

It is interesting to note that this therapeutic vaccine approach constitutes a novel therapeutic solution for treating an infection caused by a chemoresistant pathogen.

In the case of leishmaniasis, it has been shown that this therapeutic strategy proves to be effective as the current treatment, namely the administration of antiparasitics. These results are validated not only a mouse model, but also by a clinical study carried out in a naturally infected dog. Quite advantageously, the vaccine approach does not induce any resistance mechanism, unlike antibiotics. Thus, either immunity is retained over the long term and prevents reinfection, or it is possible to vaccinate the individual in the event of reinfection.

The composition of the therapeutic vaccine contains no adjuvant (other than the nanoparticles themselves), which prevents the undesirable effects associated with this type of molecule. This is advantageous since the mineral adjuvants (namely mineral salts such as aluminum salts) remain in the body for very long (several decades).

The antigens combine with the nanoparticles in solution and are delivered to the immune cells after administration, while the nanoparticles are rapidly removed (in less than 72 hours after nasal administration). The nanoparticles therefore play a dual role: stabilizing agent and antigen delivery vector.

The vaccine can be administered by oral, nasal, intra-dermal, subcutaneous or intravenous route and makes it possible to treat, with a single formulation, leishmaniasis regardless of the species of Leishmania that infected the individual. In addition, the same vaccine formulation can be administered in humans and in animals, in particular, in dogs.

### BRIEF DESCRIPTION OF THE DRAWINGS

FIG. 1 represents a Protocol for the study of Example 1.

FIG. 2 represents a SDS-PAGE (left) and PAGE in native conditions (right) of the TEL/NPL formulations carried out, after 120 h of formulation.

FIG. 3 represents a measurement of the parasitic load in the liver, spleen and bone marrow of mice, 60 days after infection of the mice.

FIG. 4 represents a protocol for the study of Example 2.

FIG. 5 represents a SDS-PAGE (left) and PAGE in native conditions (right) of the TEL/NPL formulations carried out, after 96 h of formulation.

FIG. 6 represents a representation of the parasitic load in the liver, spleen and bone marrow of mice, 90 days after infection.

FIG. 7 represents a assay of the total IgGs (on the left) and of the IgG 1 and IgG 2a (on the right) in the mice not treated and treated by chemotherapies or by s.c. and i.n. administration of the vaccine formulations.

FIG. 8 represents a re-stimulation of the splenocytes of the mice not treated and treated by chemotherapies or by s.c. and i.n. administration of the vaccine formulations, and assays of the cytokines secreted in the supernatant.

FIG. 9 represents a protocol for the study of example 3.

FIG. 10 represents a PAGE in native conditions of the TEL/NPL formulations.

FIG. 11 represents a measurement of the parasitic load in the mouse serum, not vaccinated and vaccinated by the TEL or TEL/NPL formulations, by nasal (A) or subcutaneous (B) formulations. Measurement of the parasitic load in the liver (C) and the spleen (D) of mice, not vaccinated and vaccinated by the TEL or TEL/NPL formulations.

FIG. 12 represents a protocol for the study of Example 4.

FIG. 13 represents the analysis of the parasite load of L. infantum by qPCR in the organs of the infected mice (n=5), then treated by different conditions. One-way ANOVA #p<0.05, **p<0.01, ****p<0.0001.

FIG. 14 represents the analysis of the L. infantum parasitic load by qPCR in the bone marrow of the infected mice (n=5), treated by different conditions then reinfected for 30 days. One-way ANOVA *p<0.05.

FIG. 15 represents a protocol for the study of Example 5.

FIG. 16 represents the analysis of the parasitic load of L. donovani by qPCR in the spleens of the infected mice (n=10), treated by chemotherapy. One-way ANOVA, **p<***p<0.001.

FIG. 17 represents a protocol for the study of Example 6.

FIG. 18 represents the course of the cutaneous infection in dogs treated with miltefosine and/or vaccinated, after one month.

FIG. 19 represents the course of the parasitic load in the bone marrow of dogs treated with miltefosine and/or vaccinated, after one month.

FIG. 20 represents the course of the clinical score of dogs treated with miltefosine and/or vaccinated.

### DETAILED DESCRIPTION

A first object of the present disclosure relates to a therapeutic vaccine intended for the treatment of an individual carrying a peripheral disease or an intracellular pathogen that cannot pass the blood-brain barrier, comprising at least one cationic nanoparticle consisting of a cationic polysaccharide core and at least one specific antigen, wherein the antigen is specific to a pathogen or disease that does not affect the central nervous system.

“Therapeutic vaccine” means an immunostimulating composition intended to stimulate the immune system to generate a specific immune response capable of treating individuals already suffering from the disease. This embodiment opposes that of prophylactic vaccines that are used in people who have not yet contracted the disease. The immunostimulating composition may also be called an “immunotherapeutic composition.”

“Infected individual” means a human or animal carrying a pathogen responsible for an infection not reaching the central nervous system.

“Peripheral disease” means a disease that does not affect (does not harm) the brain of the infected individual. The disease affects only metabolic systems, that is to say all organs with the exception of the brain (central nervous system). Within the meaning of the present disclosure, the diseases may be cancers that do not affect the brain or any other peripheral disease such as an autoimmune disease.

“Pathogen that cannot pass the blood-brain barrier” means a pathogen that does not infect the central nervous system, the infection therefore not affecting the brain of the infected individual.

Within the meaning of the present disclosure, the pathogen can be:
A virus not having central tropism, such as, for example: herpes simplex virus (HSV), human immunodeficiency virus (HIV), cytomegalovirus (CMV), Epstein-Barr virus (EBV).
An intra-cellular bacterium, such as: Salmonella typhi, Neisseria gonorrhoeae, Legionella, Rickettsia, Chlamydia, Chlamydophila spp, Bartonella henselae, Francisella tularensis, Listeria monocytogenes, Brucella, mycobacterium, Nocardia, Rhodococcus equi, Yersinia.
An intra-cellular parasite such as: Apicomplexans (Plasmodium spp., Cryptosporidium parvum), Trypanosomatidae (Trypanosoma cruzi, Leishmania sp.).
A fungus, such as: Histoplasma capsulatum, Cryptococcus neoformans, Sporothrix spp, etc.

“Cationic nanoparticle consisting of a cationic polysaccharide core” means a solid nanoparticle (NP) comprising a cationic polysaccharide core. The NP may or may not be crosslinked. Its core may or may not be loaded with an anionic phospholipid. This NP is not surrounded by any phospholipid layer.

“Antigen” means an antigenic protein, a mixture of antigenic proteins, or a partial or total extract of pathogen. The pathogen extract may contain proteins, polysaccharides and lipids and nucleic acids. The protein may be hydrophilic or lipophilic. The antigens can be purified, alone or in combination.

In a preferred embodiment, the mixture of antigenic proteins is composed of one or more purified antigens or a pathogen extract. The pathogen extract may be a total extract or a partial extract. In a fully preferred embodiment, the antigen is a complex protein extract obtained from an entire pathogen.

In a particular embodiment, the antigen is specific for leishmaniasis.

In a particular embodiment, the antigen is specific to the genus Leishmania, such as Leishmania infantum, Leishmania donovani or Leishmania major. In a preferred embodiment, the antigen is specific to the strain Leishmania infantum.

In the case of Leishmaniasis, it is considered that immunity that is triggered by the therapeutic vaccine may be cross-immunity by inducing a memory T response capable of recognizing different species of leishmania.

In this context, individuals treated with the therapeutic vaccine produced with the strain Leishmania infantum are immunized against all other strains of Leishmania. In addition, there is species cross-immunity, namely that the vaccine prepared from a pathogen infecting a species may potentially be used to protect other species of mammals, for example. This approach therefore allows cross-vaccination, that is to say a wide spectrum, since the vaccine is effective on all the leishmaniasis strains, whether they are cutaneous, visceral or mucosal strains.

One feature of the vaccine according to the present disclosure is based on combining the antigen with cationic NP.

In a first particular embodiment, the cationic polysaccharide forming the core of the NP is a crosslinked polymer obtained by the reaction between a polysaccharide selected from starch, dextran, dextrin, and maltodextrin, polyfructoses (inulin), polymannoses, polygalactoses, polygalactomannans (guar gum) and at least one cationic ligand selected from a primary, secondary, tertiary or quaternary ammonium, then the addition of a crosslinking agent. The crosslinking agent is chosen from epichlorohydrin, a dicarboxylic acid or an acyl chloride, such as sebacic acid. The core is not loaded with lipids.

In a preferred embodiment, the cationic polysaccharide is obtained by the reaction between maltodextrin and glycidyltrimethylammonium.

In a third particular embodiment, the cationic polysaccharide forming the nucleus of the NP is loaded with an anionic phospholipid. This anionic phospholipid can be chosen from diacyl phosphatidylglycerol, diacyl phosphatidylserine or diacyl phosphatidylinositol. In another preferred embodiment, the anionic phospholipid is dipalmitoylphosphatidylglycerol (DPPG).

In an entirely preferred embodiment, NP is a nanoparticle of maltodextrin loaded with DPPG.

In a preferred embodiment, the antigen is a complex extract obtained from a partial or total extract of a parasite responsible for a disease linked to a parasite, the infection of which does not reach the CNS. In a particular embodiment, it involves a chemoresistant parasite.

In a particular embodiment, the therapeutic vaccine induces cross-immunity of species between humans and animals. In an even more particular embodiment, it induces protective cross-immunity in humans or non-human mammals such as: canines; felines; leporidae; bovines; rodents; non-human primates; equines.

A second object of the present disclosure relates to a vaccine composition comprising a cationic nanoparticle consisting of a cationic polysaccharide core and an antigen specific to a pathogen that cannot pass the blood-brain barrier or a peripheral disease for use in the treatment of diseases stemming from a pathogen that cannot pass the blood-brain barrier or a peripheral disease.

The animals infected with Leishmaniasis are mammals such as, for example, canines, rodents, leporidae, equines, bovines, primates.

A third subject of the present disclosure relates to a composition comprising a cationic nanoparticle consisting of a cationic polysaccharide core and a specific antigen of the genus Leishmania infantum for use as a therapeutic vaccine in the treatment of cutaneous, visceral or mucosal Leishmaniasis.

In a preferred embodiment, the composition used as a therapeutic vaccine is administered nasally.

Without departing from the scope of the present disclosure, the mode of administration can also be administered mucosally (orally) or intra-dermally, subcutaneously, intravenously.

### EXAMPLES

### Abbreviations

NPL: Lipid maltodextrin nanoparticles;
TEL: total extracts of leishmania;
Glu+Allo: Glucantime+Allopurinol;
i.d: Intradermal;
i.n.: Intranasal;
i.p.: Intraperitoneal;
i.v.: Intravenous;
PRO: promastigote;
AMA: amastigote;
W: week

Example 1: Study of the feasibility of a therapeutic vaccine via a nasal route, using total extracts of Leishmania and maltodextrin nanoparticles, compared with the reference antiparasitic treatment (Glucantime+Allopurinol).

1-A Materials and Methods

a) Experimental Model:

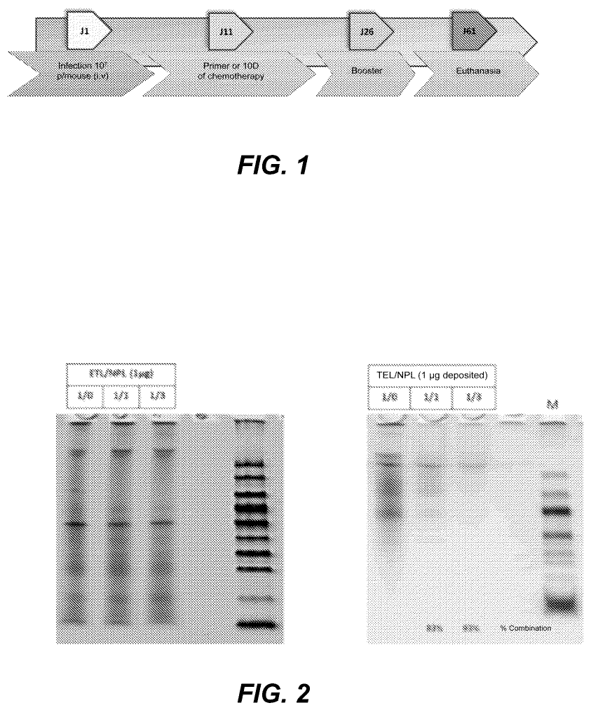

Female Balb/c mice, 8 weeks old, were infected with 1.2.10⁷ amastigotes of the strain L. infantum injected i.v. (FIG. 1). The first vaccinal and antiparasitic treatments were carried out 10 days later (D11). The drug treatment lasted 10 days (until D21). The vaccine booster was carried out 15 days after the primary (D26). Finally, the mice were euthanized 35 days later (D61).

Liver cells, spleen and bone marrow were lysed to extract DNA. The cytochrome b gene of the parasite was used to estimate the parasitic load by qPCR. Each picogram of parasitic DNA corresponds to 10 parasites. The number of parasites is estimated in 50 ng of total DNA. This protocol is shown in FIG. 1.

b) Groups and Doses Used:

Each group consisted of 8 mice, distributed as follows:
Group 1: Untreated infected control
Group 2: Chemotherapy (Glucantime+Allopurinol)
Group 3: NPL/TEL vaccine, s.c.
Group 4: NPL/TEL vaccine, i.n.

The doses administered were:
NPL/TEL s.c.: 20 μg TEL+60 μg NPL in 100 μL
NPL/TEL i.n.: 20 μg TEL+60 μg NPL in 20 μL
Chemotherapy: GLUCANTIME® 100 mgSb/kg/d by i.p. injection+Allopurinol 10 mg/kg/d orally, for 10 successive days, in 100 μL.

c) Vaccine Formulations

The formulations were produced, from total extract derived from the amastigote and promastigote forms, of a mixture of canine and human strains of L. infantum, and NPL.

1-B Results

a) Characterization of the combination of antigens with NPL

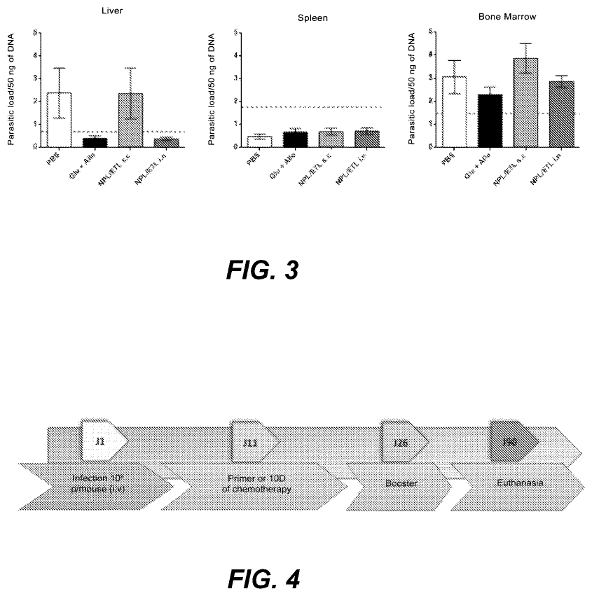

The NPLs were characterized by SDS-PAGE and native PAGE analysis. It can be seen that, in the ratio 1/3 (weight/weight), 93% of the protein antigens are combined in the nanoparticles (FIG. 2). The results are shown in FIG. 3.

b) Parasitic load at D61

It is observed at D61, an identical decrease in the parasite load in the groups immunized with intravenous NPL TEL and chemotherapy (FIG. 3). In the liver, all the parasites are eliminated (it is observed that the value is under the detection threshold). On the other hand, no improvement could be observed in the NPL TEL subcutaneous groups.

Conclusion:

These results suggest that the nasal therapeutic vaccine is as effective in reducing the parasite load as the reference treatment in this study.

Example 2: Study of the efficacy of a therapeutic nasal vaccine directed against the Leishmaniosis visceral in mice, composed of total extract of Leishmania and maltodextrin nanoparticles, and associated immune response.

2-A Materials and Methods

a) Experimental Model:

Balb/c mice, 8 weeks old, were infected with 1,108 amastigotes of the strain L. infantum injected i.d. (FIG. 4). The first vaccinal and antiparasitic treatments were carried out 10 days later (D11). The drug treatment lasted 10 days (until D21). The vaccine booster was carried out 15 days after the primary (D26). Finally, the mice were euthanized 64 days later (D90).

Liver cells, spleen and bone marrow were lysed to extract DNA. The cytochrome b gene of the parasite was used to estimate the parasitic load by qPCR. Each picogram of parasitic DNA corresponds to 10 parasites. The number of parasites is estimated in 50 ng of total DNA.

b) Groups and Doses Used:

Each group consisted of 8 mice, distributed as follows:
Group 1: Untreated infected control
Group 2: Chemotherapy (GLUCANTIME®+Allopurinol)
Group 3: NPL/TEL vaccine, s.c.
Group 4: NPL/TEL vaccine, i.n.

The doses administered were:
NPL/TEL s.c.: 20 μg TEL+60 μg NPL in 100 μL
NPL/TEL i.n.: 20 μg TEL+60 μg NPL in 20 μL
Chemotherapy: GLUCANTIME® 100 mgSb/kg/d by i.p. injection+Allopurinol 10 mg/kg/d orally, for 10 successive days, in 100 μL.

c) Vaccine Formulations

The formulations were prepared from total extract from amastigotes of canine strains of L. infantum and NPL.

In this study, the test was carried out intradermally (i.d.) to mimic biting by phlebotomine flies, the natural vectors of the infection.

2-B Results

a) Characterization of the Combination of Antigens with NPL:

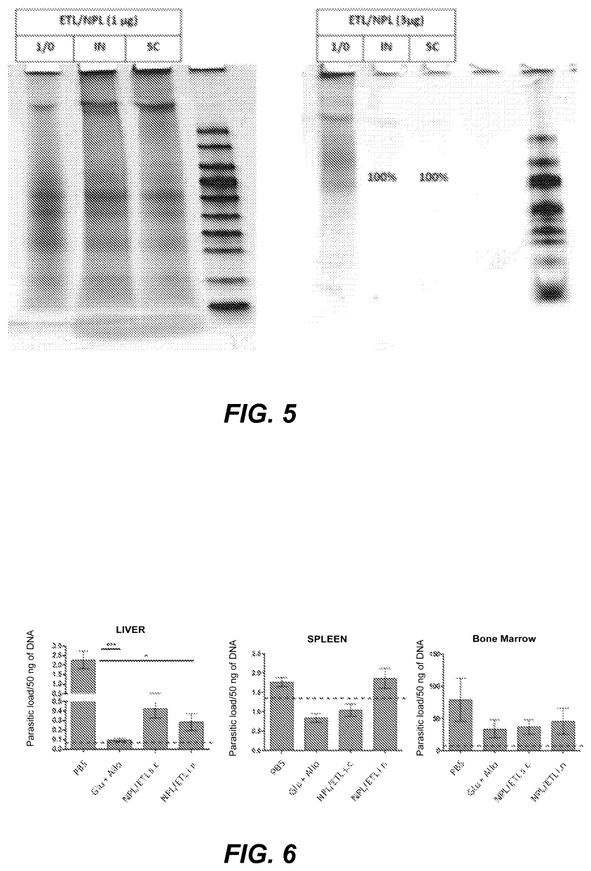

The NPLs were characterized by SDS-PAGE and native PAGE analysis. It can be seen that, in the ratio 1/3 (w/w), 100% of the protein antigens were combined in the nanoparticles (FIG. 5). The results are shown in FIGS. 6, 7, and 8.

b) Parasitic Load at D90

It was observed that after 90 days (FIG. 6), the parasitic load had decreased in the liver, the spleen and the MO of mice treated with the vaccine subcutaneously, and in the liver and the MO of mice treated by the nasal vaccine.

c) Humoral and Cellular Response:

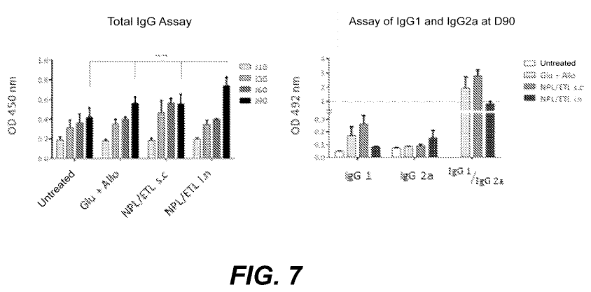

After serum assaying of the total IgGs, it was observed that the administration of the vaccine formulations via the nasal route improves the humoral response relative to the subcutaneous route and reference drug treatment (FIG. 7). Furthermore, only the nasal route allowed a ratio IgG1/IgG2a<1, showing a Th1 response necessary to combat the infection of Leishmania.

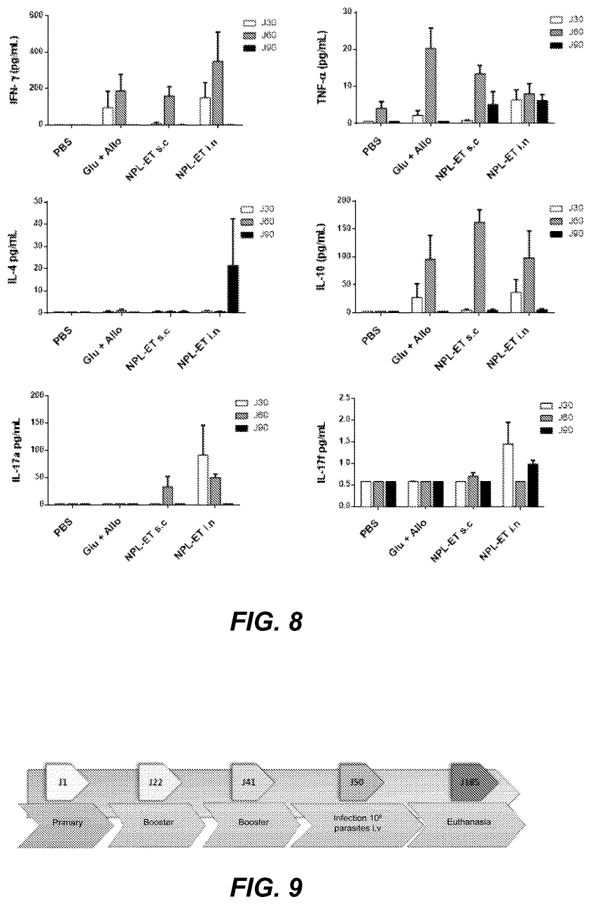

After restimulating the splenocytes, a significant secretion of the cytokines of the Th1 (INF-y) and Th17 (IL-17a and IL-17f) routes were observed from the nasally vaccinated mice (FIG. 8).

Conclusion:

The therapeutic vaccinal treatment makes it possible to reduce the parasitic load of the infected animals, in a manner similar to that of the antiparasitic treatment.

This decrease observed with the vaccine treatment is directly correlated with the induction of a Th1 immune response, both humoral and cellular.

This memory response should also protect the animals from a future reinfection in the parasite, which makes this treatment highly attractive to treat the infected animals.

Example 3: Study of the feasibility of a prophylactic vaccine using the total extracts of Leishmania and maltodextrin nanoparticles.

3-A Materials and Methods

a) Experimental Model:

Female Balb/c mice, 8 weeks old, were vaccinated 3 times nasally or subcutaneously, 20 days apart (D1, D22 and D41). They were subsequently infected, 9 days after the last administration (D50), by 106 promastigotes of the strain L. donovani injected i.v., then euthanized 135 days later (D185, FIG. 9).

b) Groups and Doses Used:

Each group consisted of 8 to 10 mice, distributed as follows:
Group 1: Saline solution i.n. (20 μL)
Group 2: Saline solution s.c. (50 μL)
Group 3: TEL i.n (10 μg TEL in 20 μL)
Group 4: TEL s.c. (10 μg TEL in 50 μL)
Group 5: NPL/TEL i.n (10 μg TEL+30 μg NPL in 20 μL)
Group 6: NPL/TEL s.c. (10 μg TEL+30 μg NPL in 50 μL)

Blood samples were taken at D64, D110, D149, and D184 to analyze the parasite load in the blood. This was measured by qPCR on the DNA of the kinetoplast of the parasites.

Mice were sacrificed by cervical dislocation after 184 days. Upon euthanasia of the mice, the livers and the spleens were collected in order to determine the parasitic load in each of these organs.

c) Vaccine Formulations

The formulations were prepared from total extract of L. donovani LV9 and NPL.

3-B Results

a) Characterization of the Combination of Antigens with NPL:

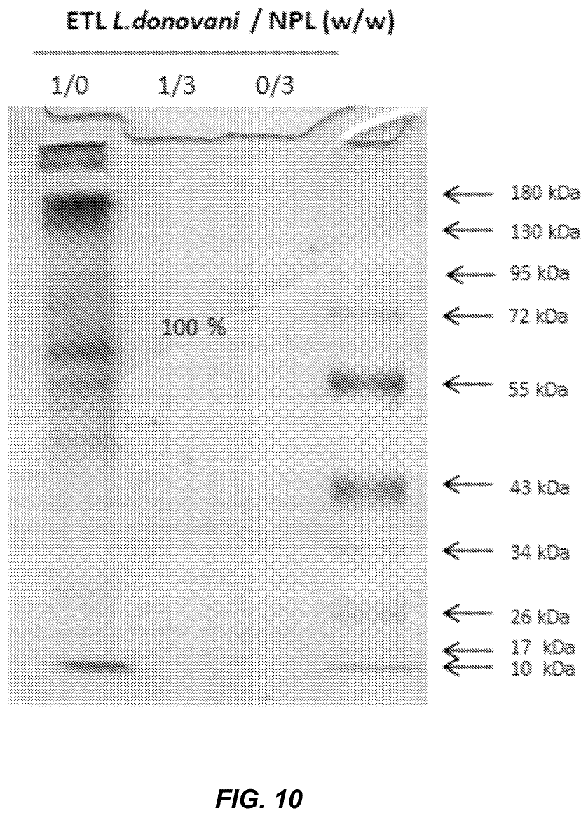

The NPL were characterized by PAGE analysis in native conditions. It can be seen that when the ratio is 1/3 (weight/weight), 100% of the protein antigens are combined in the nanoparticles (FIG. 10).

b) Parasitic Load at D185

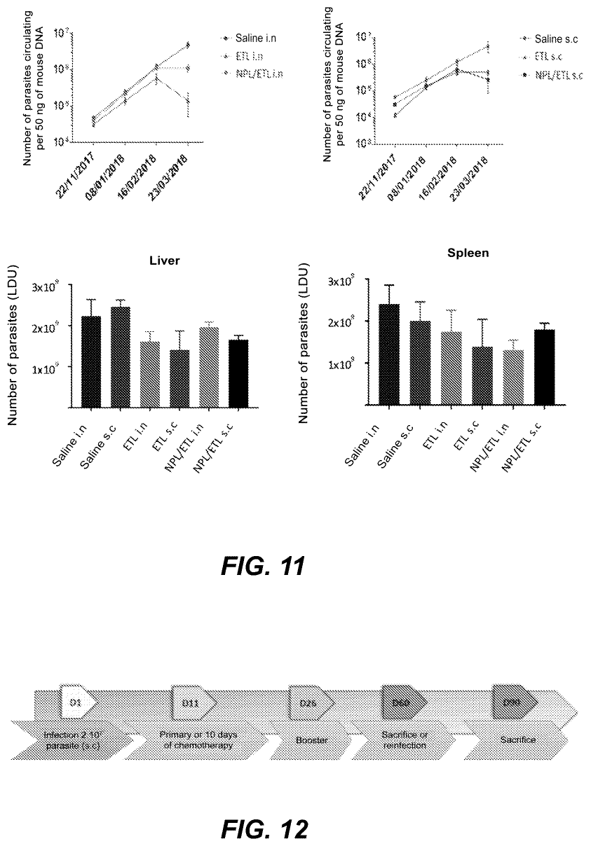

The administration of vaccine formulations substantially reduces parasitic load in blood, liver and mouse spleen (FIG. 11). The latter however remains high (>108 parasites in the organs), despite 3 prophylactic administrations of total extract combined or not combined with NPL, suggesting that this strategy does not make it possible to protect against infection.

Conclusion:

The CANILEISH® prophylactic vaccine currently on the market remains more competitive, with significant results obtained with a single administration.

Example 4: Evaluation of the efficacy of a therapeutic vaccine against infection with a L. infantum in a mouse model using antigens derived from promastigote or amastigote form of L. infantum.

4-A Experimental Protocol:

60 Balb/c mice were infected with 2.107 L. infantum by subcutaneous injection (s.c.), in promastigote or amastigote form. After 10 days, the mice were then distributed in groups of 10, and treated as follows:
Group 1: Untreated
Group 2: Reference chemotherapy (Glucantime+Allopurinol)—from D11 to D21
Group 3: NPL/TEL PRO vaccine s.c.—Primary D11 and booster D26-20 μg per dose
Group 4: NPL/TEL PRO vaccine i.n.—Primary D11 and booster D26-20 μg per dose
Group 5: NPL/TEL AMA vaccine s.c.—Primary D11 and booster D26-20 μg per dose
Group 6: NPL/TEL AMA vaccine i.n.—Primary D11 and booster D26-20 μg per dose

After 60 days, 5 mice were euthanized and the parasitic load was measured in the liver, spleen and bone marrow (FIG. 1). The remaining 5 mice were reinfected with 2.107 parasites, and their parasitic load were measured 30 days later.

This protocol is summarized in FIG. 12.

4-B Results

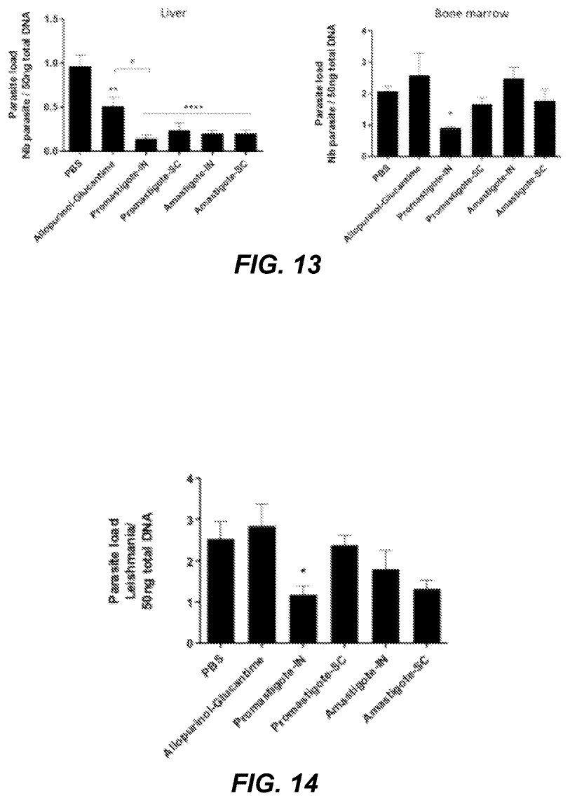

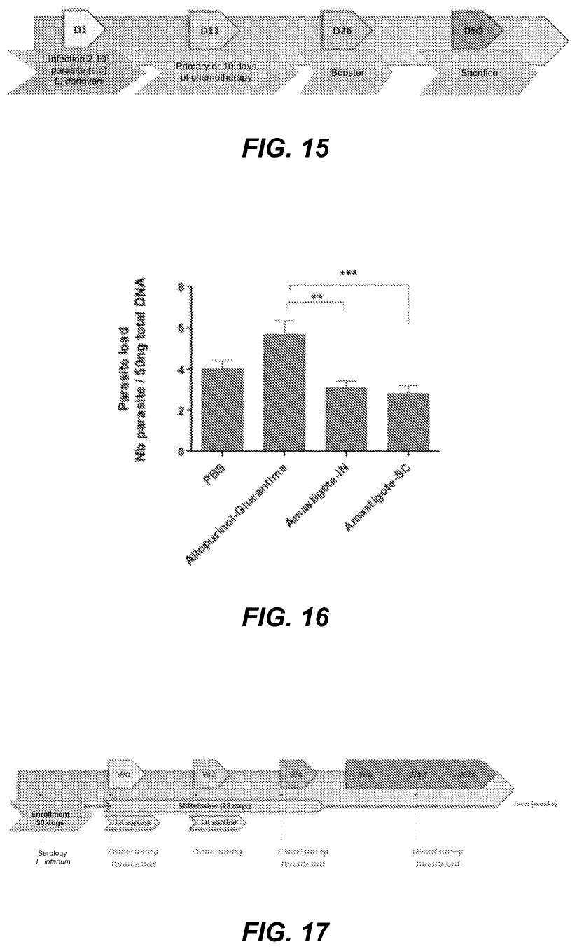

The results are presented in FIGS. 13 and 15.

The parasitic load analyses at D60 revealed a significant decrease in parasitic load in the liver of the mice treated by chemotherapy, as well as by vaccines, administered via i.n. and s.c., and regardless of the antigen source (promastigotes or amastigotes, FIG. 13). The NPL/TEL formulation made from promastigotes and administered via i.n. reduced was significantly more effective than the reference chemotherapeutic treatment.

Furthermore, a significant reduction in the parasitic load in the bone marrow was observed, only in the groups of mice treated with the NPL/TEL formulation made from promastigotes and administered i.n.

No significant variation was observed in the animal spleen.

After 90 days, a significant decrease in the parasitic load was observed, in the bone marrow of the mice treated with the NPL/TEL formulation made from promastigotes and administered i.n. (FIG. 14). No significant variation could be observed in the livers and spleens of the animals.

This study makes it possible to conclude as to the effectiveness of the NP/TEL formulations in the development of a therapeutic vaccine against Leishmaniasis. In addition, the use of promastigotes as an antigen source seems to be an interesting strategy for efficiently treating animals.

Example 5: Evaluation of the efficacy of a therapeutic vaccine against infection with a L. donovani in a mouse model using antigens derived from amastigote form of L. infantum (cross-vaccination)

5-A Experimental Protocol:

40 Balb/c mice were infected with 2.107 L. donovani, by subcutaneous injection (s.c.). After 10 days, the mice were then distributed in groups of 10, and treated as follows:
Group 1: Untreated
Group 2: Reference chemotherapy (Glucantime+Allopurinol)—from D11 to D21
Group 3: NPL/TEL vaccine s.c.— Primary D11 and booster D26-20 μg per dose
Group 4: NPL/TEL vaccine i.n.— Primary D11 and booster D26-20 μg per dose

After 90 days, the mice were euthanized and the parasitic load was measured in the liver, spleen and bone marrow.

5-B Results

The results are shown in FIG. 16.

After 90 days, a significant decrease in the parasitic load of L. donovani was observed, in the spleen of the mice treated with the NPL/TEL formulation, made from amastigotes of L. infantum and administered i.n. and s.c. (FIG. 5).

This makes it possible to conclude as to the effectiveness of the formulations NP/TEL in the development of a therapeutic vaccine, via the nasal and/or subcutaneous route, targeting different strains of the parasite. These results are promising from the perspective of developing a broad spectrum therapeutic vaccine against this parasite.

Example 6: Evaluation of the efficacy of a therapeutic vaccine against infection with a L. infantum using antigens derived from the killed target parasite, in dogs naturally infected by the parasite and compared with dogs treated by the reference chemotherapy.

6-A Experimental Protocol:

30 mixed-breed dogs naturally infected by (at least) L. infantum, and at stage II of the infection (Leishvet.org) were selected. The dogs were distributed randomly in 3 groups of 10 dogs, according to their treatment:
Group 1: Miltefosine (2 mg/kg/day for 28 days)
Group 2: NPL/TEL vaccine i.n.— Primary W0 and booster W4-100 μg per dose
Group 3: Combination of Miltefosine+Vaccine NPL/TEL i.n.

The skin infection was evaluated by microscopic analysis of skin biopsies, at W0 and W4. The presence of a parasite confirms the cutaneous infection.

The clinical score of the animals was evaluated at W0, W2, W4, W6 by taking into account the systemic signs (lymphadenopathy, apathy, diarrhea), cutaneous and mucosal signs (alopecia, hyperkeratosis, pyoderma, ulcer, vasculitis, onychogryphosis, nodules) and ocular signs (conjunctivitis, keratitis). For each parameter, a score is assigned (from 0 to 4), depending on the severity of the observed condition. The clinical score is the sum of these different scores.

The parasitic load was evaluated at W0 and W4, by qPCR analysis of a bone marrow sample at the sternum.

6-B Results

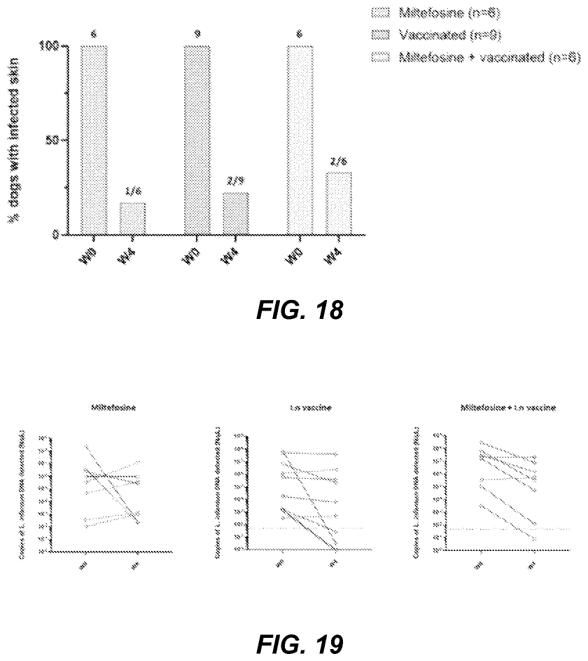

The results are presented in FIGS. 18, 19 and 20.

In group 1 (Miltefosine)—10/10 dogs had visceral leishmaniasis (VL), and 6/10 had skin damage (CL).

In group 2 (Nasal vaccine)—10/10 dogs had visceral leishmaniasis (VL), and 9/10 had skin damage (CL).

In group 3 (Miltefosine+nasal vaccine)—10/10 dogs had visceral leishmaniasis (VL), and 6/10 had skin damage (CL).

a) Skin Infection

In group 1, 5 of the 6 dogs infected and treated with Miltefosine no longer have parasites detected in the skin; in group 2, 7 of the 9 infected and vaccinated dogs no longer parasites detected in the skin; in the group 3, 4 of the 6 infected, treated and vaccinated dogs are no longer detected in the skin. The nasal vaccine seems at this stage to also be effective for treating cutaneous leishmaniasis than chemotherapy (FIG. 18).

b) Parasitic Filler

In the group treated with Miltefosine, 3 dogs have a reduction in the parasitic load, while 5 dogs have an increase, and one dog died due to the toxicity of the treatment (renal failure). In the vaccinated group, 8 dogs have a decrease in the parasite load (4 of which appear to have achieved parasite clearance), while 2 dogs have a slight increase. Finally, in the treated and vaccinated group, 6 dogs have a reduction in the parasitic load (one seems to have achieved clearance), while one dog has an increase in infection, and 2 do not show any sensitive variation (FIG. 19).

At this stage, the nasal vaccine seems to be more effective than the chemotherapy in order to reduce the parasitic load in the bone marrow.

c) Clinical Score

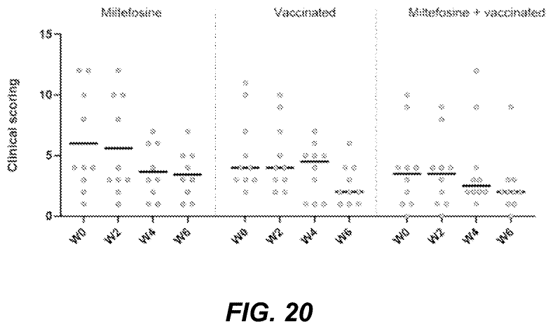

In the 3 groups studied, a notable decrease in the overall clinical score may be observed. The vaccine is as effective as chemotherapy in the improvement of the general state of health of the animals (FIG. 20).

This study demonstrates that vaccination is at least as effective as Miltefosine to treat dogs infected with L. infantum in terms of cutaneous and visceral Leishmaniasis. Furthermore, the two nasal administrations did not lead to side effects and are well tolerated. On the other hand, the 28 oral administrations of Miltefosine caused significant side effects (diarrhea, vomiting, renal failure).

1. A therapeutic vaccine for treating an individual carrying a disease or an intracellular pathogen that does not affect the central nervous system, comprising at least one cationic nanoparticle including a cationic polysaccharide core and a specific antigen, wherein the antigen is specific to the pathogen or disease.

2. The therapeutic vaccine of claim 1, wherein the specific antigen is from a pathogen responsible for leishmaniasis.

3. The therapeutic vaccine of claim 2, wherein the antigen is specific to a strain Leishmania infantum.

4. The therapeutic vaccine of claim 1, wherein the specific antigen is a tumor antigen.

5. The therapeutic vaccine of claim 1, wherein the cationic polysaccharide core is crosslinked.

6. The therapeutic vaccine of claim 1, wherein the cationic polysaccharide core is loaded with an anionic phospholipid.

7. The therapeutic vaccine of claim 6, wherein the anionic phospholipid is selected from diacyl phosphatidylglycerol, diacyl phosphatidylserine or diacyl phosphatidylinositol.

8. The therapeutic vaccine of claim 7, wherein the anionic phospholipid is dipalmitoylphosphatidylglycerol.

9. A vaccine composition comprising a cationic nanoparticle consisting of a cationic polysaccharide core and a pathogen-specific antigen that cannot pass a hematoencephalic barrier or a peripheral disease, for use in the treatment of diseases stemming from the pathogen or the disease.

10. The vaccine composition of claim 9, wherein the antigen is from the species Leishmania.

11. The vaccine composition of claim 10, wherein the antigen is from the genus Leishmania Infantum.

12. The vaccine composition of claim 9, wherein the antigen is from a parasite.

13. The vaccine composition of claim 9, wherein the antigen is from a virus.

14. The vaccine composition of claim 9, wherein the antigen is from a bacterium.

15. The vaccine composition of claim 9, wherein the antigen is from a fungus.

16. The vaccine composition, wherein the vaccine composition is in a form suitable for mucosal or injectable administration.

17. A method of treating a patient infected with a disease or an intracellular pathogen that does not affect the central nervous system, comprising administering to the patient a therapeutically effective amount of a substance comprising at least one cationic nanoparticle including a cationic polysaccharide core and a specific antigen, wherein the antigen is specific to the pathogen or disease, thereby treating the disease or pathogen in the patient.

18. The method of claim 17, wherein the antigen is from the species Leishmania.

19. The method of claim 18, wherein the antigen is from the genus Leishmania Infantum.

20. The method of claim 17, wherein the cationic polysaccharide core is crosslinked
